(19) United States
(12) Patent Application Publication
(10) Pub. No.: US 2014/0220135 A1
(43) Pub. Date: Aug. 7, 2014

(54) PERMEATION ENHANCED ACTIVE-CARRYING NANOPARTICLES

(71) Applicant: Midatech Limited, Abingdon (GB)

(72) Inventors: Thomas Rademacher, Oxfordshire (GB); Phillip Williams, Oxfordshire (GB)

(21) Appl. No.: 14/172,165

(22) Filed: Feb. 4, 2014

(30) Foreign Application Priority Data
Feb. 5, 2013 (GB) 1301991.4

(51) Int. Cl. A61K 47/48

(52) U.S. Cl. CPC A61K 47/48861
USPC 424/489; 514/5.9; 514/7.2; 514/6.8

(57) ABSTRACT

Nanoparticles having a core and a corona of ligands covalently linked to the core, wherein an active agent and a permeation enhancer are bound to or associated with the nanoparticles.

### FIELD OF THE INVENTION

The present invention relates to active-carrying nanoparticles, particularly for use in medicine, and includes methods for treatment of disorders, e.g., of blood glucose regulation.

### BACKGROUND TO THE INVENTION

The present invention is directed at compositions and products, and methods of making and administering such compositions and products, including for the treatment of mammals and particularly humans.

Bioactive agents, such as peptides, frequently suffer from poor stability, particularly thermo-stability, which may limit the conditions to which the agents can be subjected during preparation, processing, storage and/or delivery. For example, insulin is widely-used in the control and treatment of, e.g., Type 1 & Type 2 diabetes mellitus. Medical preparations of insulin for human use are generally formulated with one or more preservatives and/or stabilisers. Moreover, limited gastrointestinal stability typically presents a barrier to effective oral administration of bioactive peptides, such as insulin.

WO 2011/154711, WO 2011/156711 and WO 2012/170828 (the entire contents of each of which are hereby incorporated herein in their entirety by reference for all purposes) describe peptide-carrying nanoparticles for delivery of, e.g., human insulin via a transbuccal route of delivery. WO 2011/156711 and WO 2012/170828 describe incorporation of the peptide-carrying nanoparticles in film, optionally with a permeation or penetration enhancing agent.

There remains an unmet need for compositions capable of carrying and/or stabilising bioactive peptides, including insulin, and for methods of improved delivery of such bioactive peptides to a subject. The present invention addresses these needs among others.

### BRIEF DESCRIPTION OF THE INVENTION

The present inventors have surprisingly found that the permeation enhancers tetradecyl-D-maltoside (TDM) and lysalbinic acid sodium salt can be readily bound to an active-carrying, such as a peptide-carrying, nanoparticle, as defined herein, without significant loss of bound active even after 96 hours of incubation in solution. The relative lack of displacement of a non-covalently bound active agent is surprising, and greatly facilitates the modification of active-carrying nanoparticles to enhance their ability to penetrate cellular and organ barriers and thereby to enhance delivery of therapeutically and cosmetically important agents to a mammalian subject. Without wishing to be bound by any particular theory, the present inventors believe that the active-carrying nanoparticles of the present invention provide a highly optimised platform for delivery of bioactive agents to a subject via, for example, transbuccal, transdermal and/or across a mucosal membrane, the delivery being facilitated by the bound permeation enhancer or enhancers.

Accordingly, in a first aspect the present invention provides a nanoparticle comprising:
(i) a core which includes a metal and/or a semiconductor; and
(ii) a corona including a plurality of ligands covalently linked to the core, wherein at least one of said ligands includes a carbohydrate moiety;
(iii) at least one peptide or other bioactive agent covalently or non-covalently bound to the corona; and
(iv) a permeation enhancer selected from: alkyl-D-maltoside and lysalbinic acid.

The permeation enhancer may, in some cases, be reversibly and/or non-covalently bound to the corona of the nanoparticle.

Generally, the peptide will be a bioactive peptide, i.e. capable of stimulating a physiological response in a mammalian subject. In some cases in accordance with the present invention the peptide may be selected from the group consisting of: insulin, GLP-1, IGF1, IGF2, relaxin, INSL5, INSL6, INSL7, pancreatic polypeptide (PP), peptide tyrosine tyrosine (PTT), neuropeptide Y, oxytocin, vasopressin, GnRH, TRH, CRH, GHRH/somatostatin, FSH, LH, TSH, CGA, prolactin, ClIP, ACTH, MSH, enorphins, lipotropin, GH, calcitonin, PTH, inhibin, relaxin, hCG, HPL, glucagons, somatostatin, melatonin, thymosin, thmulin, gastrin, ghrelin, thymopoietin, CCK, GIP secretin, motin VIP, enteroglucagon, IGF-1, IGF-2, leptin, adiponectin, resistin Osteocalcin, renin, EPO, calicitrol, ANP, BNP, chemokines, cytokines, adipokines and all biologically active analogues thereof. Thus, in certain cases the peptide may be capable of stimulating a reduction in blood glucose levels in a mammalian subject. For example, the peptide may comprise or consist of monomeric and/or dimeric human insulin. Furthermore, the peptide may comprise or consist of GLP-1 or an analogue thereof. Furthermore, the at least one peptide may comprise a combination of two or more peptides specified above, e.g. insulin and GLP-1, or insulin and a GLP-1 analogue.

In some cases in accordance with the present invention said alkyl-D-maltoside may be selected from the group consisting of: hexyl-β-D-maltoside, octyl-β-D-maltoside, nonyl-β-D-maltoside, decyl-β-D-maltoside, undecyl-β-D-maltoside, dodecyl-β-D-maltoside, tridecyl-β-D-maltoside, tetradecyl-β-D-maltoside and hexadecyl-β-D-maltoside. In certain cases said alkyl-D-maltoside may comprise or consist of dodecyl-β-D-maltoside or tetradecyl-β-D-maltoside.

In some cases in accordance with the present invention said carbohydrate moiety may comprises a monosaccharide and/or a disaccharide. The carbohydrate moiety may be as defined further herein, including a carbohydrate mimetic. The carbohydrate moiety may be covalently linked to the core via a linker selected from the group consisting of: sulphur-containing linkers, amino-containing linkers, phosphate-containing linkers and oxygen-containing linkers. In some cases the linker comprises an alkyl chain of at least two carbons.

In accordance with the present invention said at least one ligand comprising a carbohydrate moiety may in some cases be selected from the group consisting of: 2′-thioethyl-α-D-galactopyranoside, 2′-thioethyl-β-D-glucopyranoside, 2′-thioethyl-2-acetamido-2-deoxy-β-D-glucopyranoside, 5′-thiopentanyl-2-deoxy-2-imidazolacetamido-α,β-D-glucopyranoside and 2′-thioethyl-α-D-glucopyranoside, wherein said at least one ligand comprising a carbohydrate moiety is covalently linked to the core via its sulphur atom.

It is specifically contemplated herein that said plurality of ligands covalently linked to the core may comprise at least a first ligand and a second ligand, wherein the first and second ligands are different. For example the first and second ligands may be as follows:
(a) said first ligand comprises 2′-thioethyl-α-D-galactopyranoside and said second ligand comprises 1-amino-17-mercapto-3,6,9,12,15,-pentaoxa-heptadecanol;
(b) said first ligand comprises 2′-thioethyl-β-D-glucopyranoside or 2′-thioethyl-α-D-glucopyranoside and said second ligand comprises 5′-thiopentanyl-2-deoxy-2-imidazolacetamido-α,β-D-glucopyranoside;
(c) said first ligand comprises 2′-thioethyl-β-D-glucopyranoside or 2′-thioethyl-α-D-glucopyranoside and said second ligand comprises 1-amino-17-mercapto-3,6,9,12,15,-pentaoxa-heptadecanol; or
(d) said first ligand comprises 2′-thioethyl-2-acetamido-2-deoxy-β-D-glucopyranoside and said second ligand comprises 1-amino-17-mercapto-3,6,9,12,15,-pentaoxa-heptadecanol,
   and wherein said first and second ligands are covalently linked to the core via their respective sulphur atoms.

In some cases the first ligand may comprise a carbohydrate moiety and said second ligand a non-carbohydrate ligand. One or more of the ligands may include an amine group. In particular, the second ligand may comprise 1-amino-17-mercapto-3,6,9,12,15,-pentaoxa-heptadecanol covalently linked to the core via its sulphur atom.

As described further herein, where there different ligands are present on the nanoparticle they may be present at, e.g., certain defined ratios or ranges of ratios. For example, the first ligand and said second ligand may present on the nanoparticle in a ratio in the range of 1:40 to 40:1, 1:10 to 10:1 or even 1:2 to 2:1.

It has been found that the nanoparticles in accordance with the present invention may be provided with a variety of numbers of ligands forming the corona. For example, in some cases the corona comprises at least 5 ligands per core, e.g. between about 10 to about 1000 ligands per core or 44-106 ligands per core.

The number of peptide molecules bound per core is not particularly limited. For certain applications, it may be desirable to employ as few as 1, 2, 3 or 4 peptides per core, while in other cases the nanoparticle of the invention may comprise at least 5 or more peptide molecules bound per core.

In accordance with the present invention, the nanoparticle core may in some cases comprise a metal. For example, a metal selected from the group consisting of: Au, Ag, Cu, Pt, Pd, Fe, Co, Cd, Gd, Zn or any combination thereof. Certain metal combinations and particular core compositions are described further herein.

Additionally or alternatively, the nanoparticle core may in some cases comprise a semiconductor. For example, a semiconductor selected from: cadmium selenide and zinc sulphide. The nanoparticle core may act as a quantum dot.

The nanoparticle core in accordance with the present invention may in some cases have a diameter in the range of about 0.5 nm to about 50 nm, such as about 1 nm to about 10 nm or about 1.5 nm to about 2 nm.

In accordance with the present invention said at least one peptide may comprise at least two, three, four, five or more different species of peptide. In particular, the nanoparticle may comprise insulin and GLP-1 bound to the corona of the same nanoparticle. The presence of more than one species of peptide bound to the nanoparticle may be preferred in certain settings (e.g. certain clinical settings) as compared with binding of a single species of peptide. In particular, combinations of peptides may be carried on a nanoparticle such that the peptides perform mutually beneficial or complementary functions and/or act in concert, such as in a synergistic fashion. The presence of more than one species may be used for the purpose of treating one or more conditions and for one or more therapeutic indications.

In accordance with the present invention, the nanoparticle may comprise, as an alternative to or in addition to the peptide, a non-peptide bioactive agent, e.g. a drug, such as an organic small molecule, a nucleic acid (e.g. siRNA), an antigen or adjuvant, which may be covalently coupled to the core of the nanoparticle or one or more ligands of the corona, or which may be non-covalently linked to the nanoparticle (e.g. bound to the corona by electrostatic or other non-covalent bonding interactions).

In accordance with the present invention the nanoparticle of the invention may comprise a component having a divalent state, such as a metal or a compound having a divalent state, or an oxide or salt thereof. For example, metals or metal complexes having the ability to exist in a divalent state are particularly useful. Such a component may be in the divalent state as added or may be transformed into a divalent state after addition. Oxides and salts of the divalent component are also useful and may be added directly or formed in situ subsequent to addition. Among the useful salts of the divalent component include halide salts, such as chloride, iodide, bromide and fluoride. Such divalent components may include, for example, zinc, magnesium, copper, nickel, cobalt, cadmium, or calcium, and their oxides and salts thereof. The component is desirably present in an amount sufficient to produce a stabilizing effect and/or in an amount sufficient to enhance the binding of the peptide to the corona to t level great than the level of binding of the peptide to the corona in the absence of the component having a divalent state. In some cases, the component having a divalent state is desirably present in an amount of about 0.5 to 2.0 equivalents to the core metal (e.g. gold), or optionally about 0.75 to 1.5 equivalents to the core metal (e.g. gold). In the context of the present invention, “equivalents” may be mole equivalents, for example 1.0 equivalent of zinc may be taken to mean the same number of zinc atoms or Zn²⁺ cations as the number of gold atoms in the core of the nanoparticle.

The divalent component may in some cases be present in the corona of the nanoparticle. It is specifically contemplated herein that the divalent component may be included in the nanoparticle, including in the corona of the nanoparticle as a result of inclusion of the divalent component in the process of synthesis of the nanoparticle. Additionally or alternatively, the divalent component may be added after synthesis of the nanoparticle. In some cases in accordance with the present invention, the divalent component, such as zinc may be selected from: Zn²⁺ and ZnO. For example, the zinc may be in the form of ZnCl₂.

In a further aspect the invention provides a plurality of nanoparticles of the invention. For example, a plurality may be 100, 1000, 100000, or more. The plurality may be in as associated form, a suspension or contained together in a single package, container or carrier. In certain cases, the plurality may take the form of one or more doses (e.g. a defined quantity of peptide or peptide activity units), such as in the form of a therapeutic dose or defined number of doses.

In a further aspect the present invention provides a pharmaceutical or cosmetic composition comprising a plurality of nanoparticles of the invention and one or more pharmaceutically or cosmetically acceptable carriers or excipients. In some cases, the pharmaceutical or cosmetic composition may be formulated for administration to a mammalian subject by dermal, buccal, transmucosal, intraveneous (i.v.), intramuscular (i.m.), intradermal (i.d.) or subcutaneous (s.c) route.

In a further aspect the present invention provides a method of enhancing the cellular permeability of an active-carrying nanoparticle, comprising:
(a) providing an active-carrying nanoparticle comprising:
   (i) a core which includes a metal and/or a semiconductor;
   (ii) a corona including a plurality of ligands covalently linked to the core, wherein at least one of said ligands includes a carbohydrate moiety; and
   (iii) at least one peptide or other bioactive agent covalently or non-covalently bound to the corona; and
(b) contacting the at least one active-carrying nanoparticle with a permeation enhancer selected from: alkyl-D-maltoside and lysalbinic acid under conditions which allow the permeation enhancer to bind to the corona of the nanoparticle.

The method of this aspect of the invention may further comprise separating the active-carrying nanoparticle having said permeation enhancer bound thereto from excess permeation enhancer. In some cases, said separating may comprise centrifugation (e.g. centrifugation at 15000-20000 g, such as at about 15000 g).

The active-carrying nanoparticle of this aspect of the invention may be any nanoparticle as defined in connection with the first aspect of the invention. In particular, the active-carrying nanoparticle which is contacted with said permeation enhancer may be as defined in any one of WO 2011/154711, WO 2011/156711 and WO 2012/170828, the entire contents of each of which are expressly incorporated herein by reference for all purposes.

Preferably, the active-carrying nanoparticle of this aspect of the invention comprises a peptide non-covalently bound to the corona of the nanoparticle.

In some cases in accordance with this and other aspects of the present invention said alkyl-D-maltoside may be selected from the group consisting of: hexyl-β-D-maltoside, octyl-β-D-maltoside, nonyl-β-D-maltoside, decyl-β-D-maltoside, undecyl-β-D-maltoside, dodecyl-β-D-maltoside, tridecyl-β-D-maltoside, tetradecyl-β-D-maltoside and hexadecyl-β-D-maltoside. In certain cases said alkyl-D-maltoside may comprise or consist of dodecyl-β-D-maltoside or tetradecyl-β-D-maltoside.

In a further aspect the present invention provides a method of lowering blood glucose in a mammalian subject in need thereof, comprising administering a therapeutically effective amount of a nanoparticle of the invention, for example a nanoparticle having insulin and/or GLP-1 bound to the corona and a permeation enhancer selected from: tetradecyl-D-maltoside and lysalbinic acid.

In a further aspect the present invention provides a method of treating diabetes in a mammalian subject in need thereof, comprising administering a therapeutically effective amount of a nanoparticle of the invention, for example a nanoparticle having insulin and/or GLP-1 bound to the corona and a permeation enhancer selected from: alkyl-D-maltoside and lysalbinic acid. The nanoparticle of the invention or a pharmaceutical composition comprising the nanoparticle may be administered to a subject by any suitable route of administration. In particular cases, the nanoparticle of the invention or pharmaceutical composition comprising said nanoparticle may be administered via a transdermal, transbuccal or transmucosal route of administration. In some cases said alkyl-D-maltoside may be selected from the group consisting of: hexyl-β-D-maltoside, octyl-β-D-maltoside, nonyl-β-D-maltoside, decyl-β-D-maltoside, undecyl-β-D-maltoside, dodecyl-β-D-maltoside, tridecyl-β-D-maltoside, tetradecyl-β-D-maltoside and hexadecyl-β-D-maltoside.

In a further aspect the present invention provides a nanoparticle of the invention for use in a method of medical treatment. The nanoparticle may be formulated for pharmaceutical use, for example by combining one or, typically, a plurality of nanoparticles of the invention with one or more pharmaceutically acceptable excipients or carriers. The nanoparticle of the invention or pharmaceutical composition comprising said nanoparticle may be formulated for administration by any suitable route for delivery to a subject. In particular, the nanoparticle of the invention or pharmaceutical composition comprising said nanoparticle may be formulated for administration a transdermal, transbuccal or transmucosal route of administration.

In a further aspect the present invention provides a nanoparticle of the invention (for example a nanoparticle having insulin and/or GLP-1 bound to the corona and a permeation enhancer selected from: alkyl-D-maltoside and lysalbinic acid) for use in a method of lowering blood glucose in a mammalian subject in need thereof and/or treating diabetes in a mammalian subject in need thereof.

In a further aspect the present invention provides use of a nanoparticle of the invention (for example a nanoparticle having insulin and/or GLP-1 bound to the corona and a permeation enhancer selected from: alkyl-D-maltoside and lysalbinic acid) in the preparation of a medicament for use in a method of lowering blood glucose in a mammalian subject in need thereof and/or treating diabetes.

In a further aspect the present invention provides an article of manufacture comprising:
at least one nanoparticle of the invention (for example a nanoparticle having insulin and/or GLP-1 bound to the corona and a permeation enhancer selected from: alkyl-D-maltoside and lysalbinic acid);
a container for housing said at least one nanoparticle; and
an insert and/or a label.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows a schematic representation of nanoparticles having a plurality of ligands in the ratio 1:1 of alpha-Gal:EG6NH2 “NP-alpha-Gal (1) EG6NH2 (1)”;

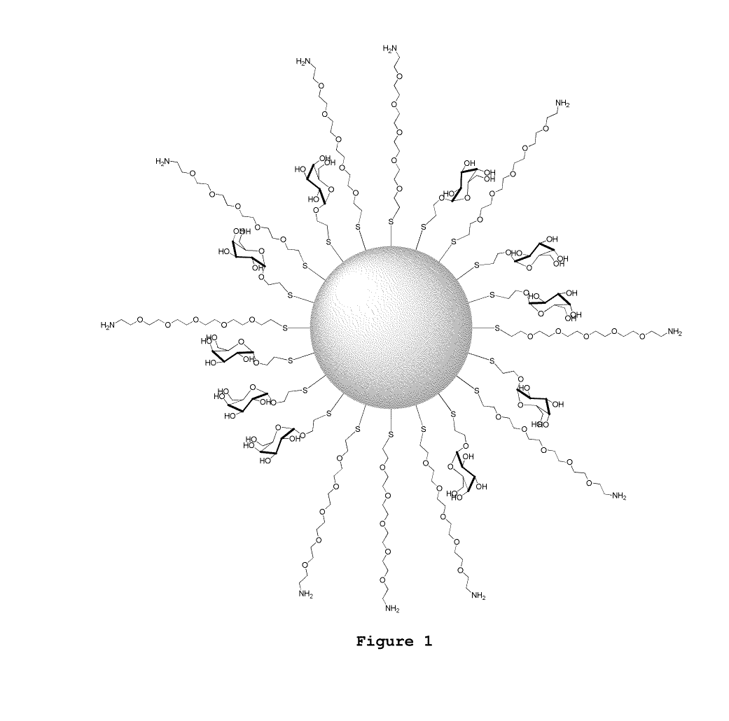

FIG. 2 shows the detected level of released insulin from nanoparticles incubated with varying concentrations of tetradecyl-D-maltoside (TDM) for 1 hour at room temperature (note the broken y-axis and the maximum insulin level detected in the absence of centrifugation);

FIG. 3 shows the detected level of released insulin from nanoparticles incubated with varying concentrations of tetradecyl-D-maltoside (TDM) for 96 hours at room temperature (note the broken y-axis and the maximum insulin level detected in the absence of centrifugation);

FIG. 4 shows the detected level of released insulin from nanoparticles incubated with varying concentrations of lysalbinic acid for 1 hour (squares) and 96 hours (circles) at room temperature (note the broken y-axis and the maximum insulin level detected in the absence of centrifugation);

FIG. 5 shows the lack of effect of lysalbinic acid (squares) and tetradecyl-D-maltoside (TDM) (circles) on insulin release for nanoparticles expressed as a percentage of insulin released (y-axis scale 0.00-0.30%) plotted against the percentage of permeation enhancer at an insulin level of 75 U/ml;

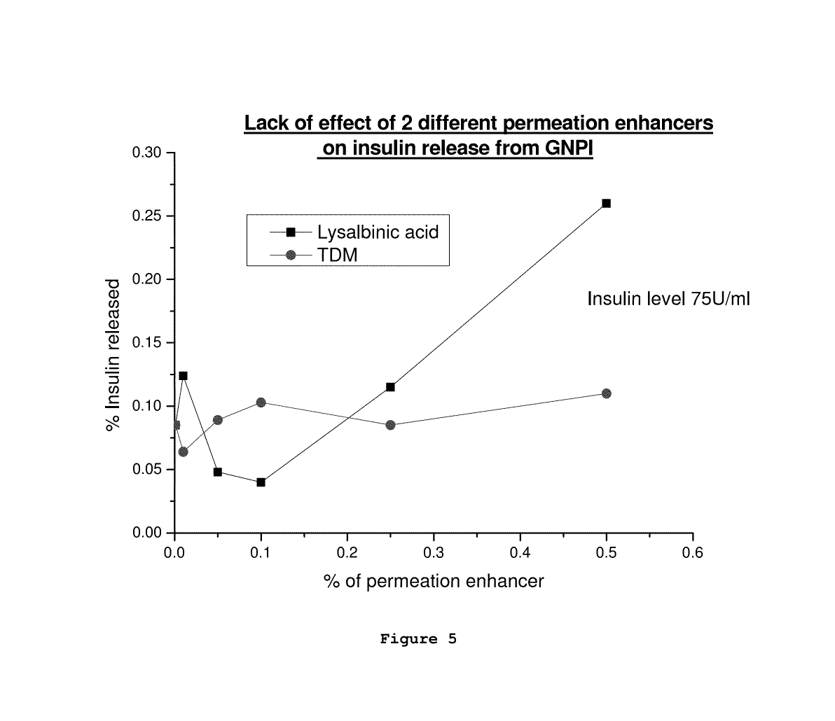

FIG. 6 shows the lack of effect of lysalbinic acid (squares) and tetradecyl-D-maltoside (TDM) (circles) on insulin release for nanoparticles expressed as a percentage of insulin released (y-axis scale 0-100%) plotted against the percentage of permeation enhancer at an insulin level of 75 U/ml;

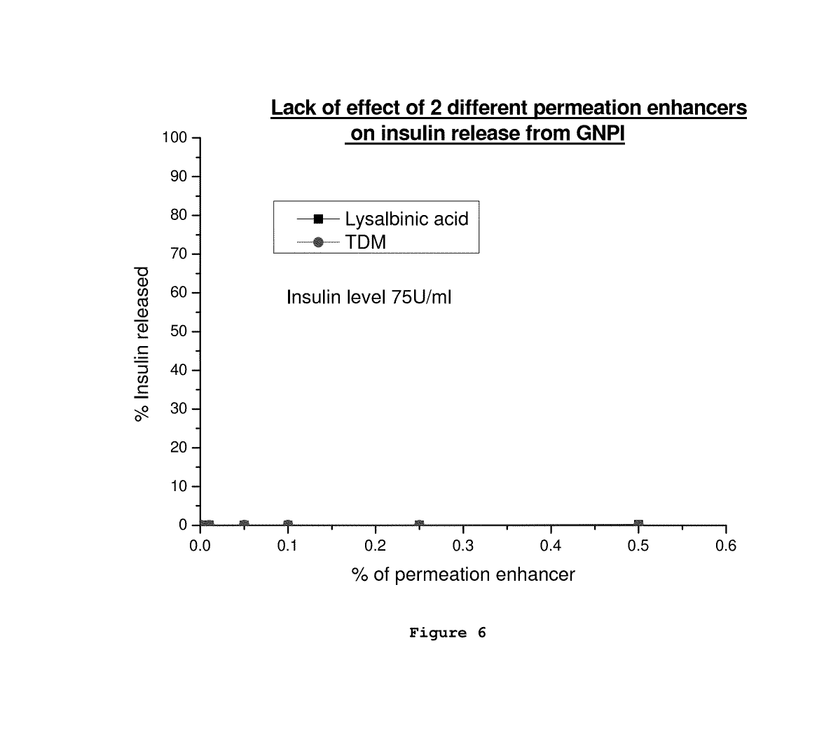

FIG. 7 shows rate constant (min⁻¹) for glucose clearance derived for each test item. Transbuccal nanoparticle-insulin (tb NP-insulin)—item 5; tb NP-insulin/dodecyl-β-D-maltoside (DoD)—item 3; tb NP-insulin plus DoD—item 4; subcutaneous (s.c.) NP-insulin—item 1; and s.c. Lispro (insulin analogue)—item 6. Error bars are shown, numbers of repeats (n) are shown, and p values are shown;

FIG. 8 shows the rate constants (min⁻¹) for glucose clearance plotted against the dosage of dodecyl-β-D-maltoside (DoD) for the test items (μg DoD/strip). A clear dose-dependence on dodecyl-β-D-maltoside is apparent.

### DETAILED DESCRIPTION OF THE INVENTION

In describing the present invention, the following terms will be employed, and are intended to be defined as indicated below.

As used herein, “nanoparticle” refers to a particle having a nanomeric scale, and is not intended to convey any specific shape limitation. In particular, “nanoparticle” encompasses nanospheres, nanotubes, nanoboxes, nanoclusters, nanorods and the like. In certain embodiments the nanoparticles and/or nanoparticle cores contemplated herein have a generally polyhedral or spherical geometry.

Nanoparticles comprising a plurality of carbohydrate-containing ligands have been described in, for example, WO 2002/032404, WO 2004/108165, WO 2005/116226, WO 2006/037979, WO 2007/015105, WO 2007/122388, WO 2005/091704 (the entire contents of each of which is expressly incorporated herein by reference) and such nanoparticles may find use in accordance with the present invention. Moreover, gold-coated nanoparticles comprising a magnetic core of iron oxide ferrites (having the formula XFe₂O₄, where X=Fe, Mn or Co) functionalised with organic compounds (e.g. via a thiol-gold bond) are described in EP2305310 (the entire contents of which is expressly incorporated herein by reference) and are specifically contemplated for use as nanoparticles/nanoparticle cores in accordance with the present invention.

As used herein, “corona” refers to a layer or coating, which may partially or completely cover the exposed surface of the nanoparticle core. The corona includes a plurality of ligands which include at least one carbohydrate moiety. Thus, the corona may be considered to be an organic layer that surrounds or partially surrounds the metallic and/or semiconductor core. In certain embodiments the corona provides and/or participates in passivating the core of the nanoparticle. Thus, in certain cases the corona may include a sufficiently complete coating layer substantially to stabilise the metal-containing core. However, it is specifically contemplated herein that certain nanoparticles having cores, e.g., that include a metal oxide-containing inner core coated with a noble metal may include a corona that only partially coats the core surface.

As used herein, “subject” is intended to encompass any mammal, such as human, companion animals (cat, dog), domestic animals (cow, pig, sheep, goat, horse), and zoo animal. Preferably, the subject is a human. In some cases, the human subject has diabetes, pre-diabetes or has been identified as having one or more risk factors for the development of diabetes.

As used herein, “peptide” is intended to encompass any sequence of amino acids and specifically includes peptides, polypeptides proteins (including proteins having secondary, tertiary and/or quaternary structure) and fragments thereof. The expression “peptide bound to” is specifically intended to encompass a part (but may include the whole) of the amino acid sequence of the peptide forming a bonding interaction with one or more parts (such as a chemical group or moiety) of one or more of the plurality of ligands of the nanoparticle. In certain embodiments the peptide may have a molecular weight of <500 kDa, <100 kDa, <50 kDa, such as up to 20 kDa.

Accordingly, in one aspect the present invention provides a nanoparticle comprising:
(i) a core which includes a metal and/or a semiconductor;
(ii) a corona which includes a plurality of ligands covalently linked to the core, wherein at least one of said ligands includes a carbohydrate moiety;
(iii) at least one peptide bound to the corona; and
(iv) a permeation enhancer selected from: alkyl-D-maltoside and lysalbinic acid.

The term “bound” is intended to include a physical and/or a chemical association between two components. This term includes any form of chemical linkage, e.g., covalent, ionic, hydrogen bonding or intermolecular forces, such as van der Waals forces or electrostatic forces. The term includes physical coupling or linking. This physical and or chemical association may be intended to be reversible, i.e., the component may be separated or disassociated, one from the other, e.g., to release the active component from the carrier component.

The peptide may be reversibly bound to the corona. In particular it is specifically contemplated that the peptide may be bound to a part of the nanoparticle non-covalently. Without wishing to be bound by any theory, it is presently believed that a peptide may participate in one or more reversible binding interactions with one or more ligands that provide the corona of the nanoparticle. In particular, a portion of the sequence of amino acids may participate in hydrogen bonding, Van der Waals forces and/or electrostatic interactions with one or more ligands (e.g. interacting with one or more functional groups of an exposed ligand). The peptide binding may involve adsorption, absorption or other direct or indirect interaction with one or more ligands of the nanoparticle.

As described herein with reference to certain embodiments of the present invention, the peptide may be bound such that at least a fraction or portion of the bound peptide is released from the nanoparticle upon contacting the nanoparticle with a physiological solution. As described herein the peptide may be bound to the nanoparticle in a manner such that the peptide is stabilised (e.g. thermostabilised) while bound, but is releasable and available in a form that is biologically active (for example, releasable such that the peptide is detectable by ELISA and/or capable of exerting at least one biological action in an in vitro or in vivo system that is characteristic of the free peptide). In particular, when the peptide includes (human) insulin, the peptide may be bound to the nanoparticle such that a suspension of the insulin-bound nanoparticles gives a positive result in an ELISA for (human) insulin and/or exerts an effect on blood glucose levels in a mammalian subject following administration thereto.

The peptide (including without limitation polypeptide, protein, or fragment thereof) may be selected from the group consisting of: insulin, GLP-1, IGF1, IGF2, relaxin, INSL5, INSL6, INSL7, pancreatic polypeptide (PP), peptide tyrosine tyrosine (PTT), neuropeptide Y, oxytocin, vasopressin, GnRH, TRH, CRH, GHRH/somatostatin, FSH, LH, TSH, CGA, prolactin, ClIP, ACTH, MSH, endorphins, lipotropin, GH, calcitonin, PTH, inhibin, relaxin, hCG, HPL, glucagons, somatostatin, melatonin, thymosin, thmulin, gastrin, ghrelin, thymopoietin, CCK, GIP secretin, motin VIP, enteroglucagon, leptin, adiponectin, resistin, osteocalcin, renin, EPO, calicitrol, ANP, BNP, chemokines, cytokines, adipokines and biologically active analogs thereof. In certain embodiments the peptide is capable of stimulating a reduction in blood glucose levels in a mammalian subject. Thus, in some cases in accordance with the present invention the peptide may include monomeric and/or dimeric human insulin.

In certain cases in accordance with the present invention there may be at least 1, at least 2, at least 3, at least 4, at least 5, at least 10 or more peptide molecules bound per core on average. There may be a single type of peptide or two or more different peptides. Where a combination of two different peptides are bound to a nanoparticle, the different peptides may in some cases be present in a ratio of 1:10 to 10:1, such as 1:2 to 2:1. Thus, complementary combinations of peptides that are advantageously co-administered are specifically contemplated.

As used herein the term “carbohydrate”” is intended to include compounds of the general formula Cₙ(H₂O)ₘ where n=m and n is greater than 3. Also, included within the definition of carbohydrate are carbohydrate analogues/mimetics that are not included in the general formula Cₙ(H₂O)ₘ. The carbohydrate analogues/mimetics include but are not limited to pseudo-sugars (carba-sugars), amino-sugars, imino-sugars and inositols. Amino-sugars include polyhydroxylated piperidines, pyrrolidines, pyrrolizidines and indolizidines.

As described herein the nanoparticle in accordance with the present invention includes a plurality of ligands covalently linked to a metal-containing core. The ligands may be the same or different. In particular embodiments, the plurality of ligands may include a first class of ligands including at least one carbohydrate moiety and a second class of non-carbohydrate ligands. As used herein the at least one ligand including carbohydrate moiety will generally include one or more sugar groups, such as a monosaccharide, a disaccharide and/or a polysaccharide and/or one or more pseudo-sugar groups (such as pseudo sugar selected from: a carba-sugar, an amino-sugar, an imino-sugar, an inositol, a polyhydroxylated piperidine, a pyrrolidine, a pyrrolizidine and an indolizidine). The ligands are covalently linked to the core of the nanoparticle. Therefore, the term “carbohydrate moiety” is to be understood to include chemical derivatives of carbohydrates such as glycosides wherein the ligand includes a sugar group or pseudo-sugar group (such as pseudo sugar selected from: a carba-sugar, an amino-sugar, an imino-sugar, an inositol, a polyhydroxylated piperidine, a pyrrolidine, a pyrrolizidine and an indolizidine) attached to a non-sugar atom or molecule. In particular cases, the ligand including a carbohydrate moiety in accordance with the present invention may include a glycoside of galactose, glucose, glucosamine, N-acetylglucosamine, mannose, fucose and/or lactose, e.g. the carbohydrate moiety may include a galactopyranoside and/or a glucopyranoside. The carbohydrate-containing ligand may be covalently linked to the core via a linker selected from sulphur-containing linkers, amino-containing linkers and phosphate-containing linkers. Combinations of linkers off of the core may also be used. The linker may in some cases include an alkyl chain of at least two carbons.

The ligand linked to the core includes one or more carbohydrate (saccharide) groups, e.g. including a polysaccharide, an oligosaccharide or a single saccharide group. The ligand may also be a glycanoconjugate such as a glycolipid or a glycoprotein. In addition to the carbohydrate group, the ligand may additionally include one or more of a peptide group, a protein domain, a nucleic acid molecule (e.g. a DNA/RNA segment) and/or a fluorescent probe.

In certain cases the particles may have more than one species of ligand immobilised thereon, e.g. 2, 3, 4, 5, 10, 20 or 100 different ligands. Alternatively or additionally a plurality of different types of particles can be employed together.

In certain cases, the mean number of ligands linked to an individual metallic core of the particle is at least 5, at least 10 or at least 20 ligands. The number may be in the range 10 to 10,000 such as 10 to 1,000, more particularly 20 to 500 or 44 to 106 ligands per core.

Preferably, substantially all of the ligands are attached covalently to the core of the particles. Protocols for carrying this out are known in the art (see, e.g. WO 2002/032404, WO 2004/108165, WO 2005/116226, WO 2006/037979, WO 2007/015105, WO 2007/122388, WO 2005/091704, WO 2011/154711, WO 2011/156711 and WO 2012/170828). This may be carried out by reacting ligands with reductive end groups with a noble metal such as gold under reducing conditions. An exemplary method of producing the particles employs thiol derivatised carbohydrate moieties to couple the ligands to particles. Thus, the ligand is derivatised as a protected disulphide. Conveniently, the disulphide protected ligand in methanol can be added to an aqueous solution of tetrachloroauric acid. A preferred reducing agent is sodium borohydride. In certain embodiments, the nanoparticles are soluble in organic solvents and in water and physiological solutions. The present inventors have found that the nanoparticles as described herein are suitable for therapeutic applications, and may be non-toxic, soluble and/or excreted in the urine.

In certain cases in accordance with the present invention, the at least one ligand comprising a carbohydrate moiety is selected from the group of: 2′-thioethyl-α-D-galactopyranoside, 2′-thioethyl-β-D-glucopyranoside, 2′-thioethyl-2-acetamido-2-deoxy-β-D-glucopyranoside, 5′-thiopentanyl-2-deoxy-2-imidazolacetamido-α, β-D-glucopyranoside and 2′-thioethyl-α-D-glucopyranoside, and wherein said at least one ligand comprising a carbohydrate moiety is covalently linked to the core via the thiol sulphur.

Additionally or alternatively, the plurality of ligands may include an amine group. Thus, a ligand comprising a carbohydrate group may include an amine group (e.g. as part of the carbohydrate, such as a glucosamine, and/or as a constituent group of a non-carbohydrate part of the ligand. Moreover, where the plurality of ligands includes at least one non-carbohydrate ligand, the non-carbohydrate group may include an amine group. The at least one non-carbohydrate ligand may include 1-amino-17-mercapto-3,6,9,12,15,-pentaoxa-heptadecanol covalently linked to the core via the thiol sulphur.

In accordance with certain embodiments of the present invention, the plurality of ligands may include said at least one ligand including a carbohydrate moiety and said at least one non-carbohydrate ligand wherein the said ligands are different and are present on the nanoparticle in a ratio of 1:40 to 40:1, such as a ratio of 1:10 to 10:1, more particularly a ratio of 1:2 to 2:1.

The nanoparticle “core” includes a metal and/or a semiconductor. Suitable cores are described in, e.g., WO 2002/032404, WO 2004/108165, WO 2005/116226, WO 2006/037979, WO 2007/015105, WO 2007/122388, WO 2005/091704, WO 2011/154711, WO 2011/156711 and WO 2012/170828 (the entire contents of each of which is expressly incorporated herein by reference) and such nanoparticle cores may find use in accordance with the present invention. Moreover, gold-coated nanoparticles including a magnetic core of iron oxide ferrites (having the formula XFe₂O₄, where X=Fe, Mn or Co) are described in EP2305310 (the entire contents of which is expressly incorporated herein by reference) and may find use in accordance with the present invention.

In some cases in accordance with the present invention the nanoparticle core includes a metal selected from the group of: Au, Ag, Cu, Pt, Pd, Fe, Co, Gd, Zn or any combination thereof. The core may include a passive metal selected from the group of: Au, Ag, Pt, Pd and Cu, or any combination thereof. In certain embodiments a specific combination of metals may be employed, such as a combination of metals selected from the group of: Au/Fe, Au/Ag, Au/Cu, Au/Ag/Cu, Au/Pt, Au/Pd, Au/Ag/Cu/Pd, Au/Gd, Au/Fe/Cu, Au/Fe/Gd, Au/Fe/Cu/Gd.

In some cases in accordance with the present invention the nanoparticle core may be magnetic. The core may include an NMR active atom, such as a metal selected from the group of: Mn²⁺, Gd³⁺, Eu²⁺, Cu²⁺, V²⁺, Co²⁺, Ni²⁺, Fe²⁺, Fe³⁺ and lanthanides³⁺.

In some cases in accordance with the present invention the nanoparticle core may include a semiconductor, such as that selected from the group of: cadmium selenide, cadmium sulphide, cadmium tellurium and zinc sulphide.

In some cases in accordance with the present invention the nanoparticle core may include a metal oxide coated with a metal selected from the group of: Au, Ag, Cu, Pt, Pd and Zn, or any combination thereof. The metal oxide may advantageously be of the formula XFe₂O₄, where X is a metal selected from the group of: Fe, Mn and Co.

In some cases in accordance with the present invention the nanoparticle core may have an average diameter in the range of about 0.5 nm to about 50 nm, such as about 1 nm to about 10 nm, more specifically about 1.5 nm to about 2 nm.

In accordance with the present invention the nanoparticle or nanoparticle-containing composition comprises at least one permeation enhancer. The permeation enhancer may be a detergent such as an alkyl-β-D-glucoside, alkyl-β-D-maltoside, alkyl-β-D-thioglucoside, or alkyl-β-D-thiomaltoside. In some cases, said alkyl chain is in the range C2-C20 (e.g. C2, C3, C4, C5, C6, C7, C8, C9, C10, C11, C12, C13, C14, C15, C16, C17, C18, C19 or C20). The alkyl chain may be branched or straight chain. In particular cases, said permeation enhancer may be an alkyl-D-maltoside selected from the group consisting of: hexyl-β-D-maltoside, octyl-β-D-maltoside, nonyl-β-D-maltoside, decyl-β-D-maltoside, undecyl-β-D-maltoside, dodecyl-β-D-maltoside, tridecyl-β-D-maltoside, tetradecyl-β-D-maltoside and hexadecyl-β-D-maltoside. In certain cases said alkyl-D-maltoside may comprise or consist of dodecyl-β-D-maltoside or tetradecyl-β-D-maltoside. In certain cases, the permeation enhancer may comprise or consist of lysalbinic acid.

The following is presented by way of example and is not to be construed as a limitation to the scope of the claims.

### EXAMPLES

### Example 1

### Preparation of Peptide-Carrying Nanoparticles Having Permeation Enhancers Bound Thereto, and Investigation of the Possible Displacement of Bound Insulin

Preparation of nanoparticles, binding of peptides, such as insulin and/or GLP-1, and characterisation of the resulting peptide-carrying nanoparticles are described in WO 2011/154711, WO 2011/156711 and WO 2012/170828, the detailed description and examples of each of which are expressly incorporated herein by reference in their entirety.

To a mix of amine-mercapto hexaethylenglycol linker and alpha-galactose ligand in a ratio 1:1 (0.58 mmol, 3 eq.) in MeOH (49 mL) was added an aqueous solution of gold salt (7.86 mL, 0.19 mmol, 0.025M). The reaction was stirred during 30 seconds and then, an aqueous solution of NaBH4 (1N) was added in several portions (4.32 mL, 4.32 mmol). The reaction was shaken for 100 minutes at 900 rpm. After this time, the suspension was centrifuged 1 minute at 14000 rpm. The supernatant is removed and the precipitated was dissolved in 2 mL of water. Then, 2 mL of the suspension were introduced in two filters (AMICON, 10 KDa, 4 mL) and were centrifuged 5 minutes at 4500 g. The residue in the filter was washed twice more with water. The final residue was dissolved in 80 mL of water.

Preparation of insulin stock solution: weight 20 mg human insulin into a clean glass vial and add 8.7 ml 10 mM HCl mix gently insulin will dissolve completely, then pH back to 7.5 by adding 1.3 ml 100 mM Tris base, the solution will go cloudy briefly as the insulin passes through its isoelectric point, check the pH is 7.5 and store capped at 4° C., this is the 2 mg/ml insulin stock solution.

Variable amounts of alphaGal (1) EG6NH2(1) NPs to an eppendorf or suitably sized vessel, for example; 15, 30, 60, 120, 240 and 480 nmoles gold content of NP, make up to a total volume of 200 μl with water, then add 50 μl of human insulin (2 mg/ml in tris HCl pH 7.5—see above for preparation of insulin stock solution). Mix gently and leave at room temp for 2 h, follow with a 2 minute bench spin (2000 rpm) to bring down the aggregate.

### Test Compounds

2 permeation enhancers were tested: tetradecyl-D-maltoside (TDM) Sigma Cat no. 15826 and lysalbinic acid sodium salt MP Biomedical Cat no. 205645.

### Experimental Details and Results

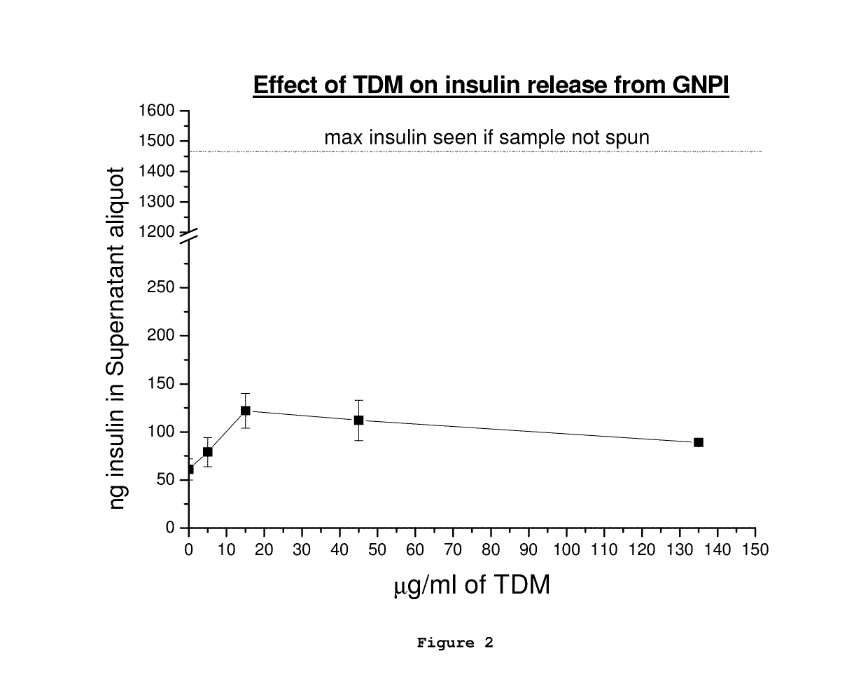

Variable amounts 2.5, 7.5, 22.5 and 67.5 μg of TDM dissolved in water were added to eppendorfs tubes these were made up to a final volume of 0.475 ml with water, the test was started by addition of 25 μl of 100 U/ml insulin-carrying gold nanoparticles (“GNPI”) (the μg/ml TDM will be double the values shown above as these are in 0.5 ml). Samples were mixed and left at RT for 1 h, they were then spun for 2 min at 18000 g and 10 μl of each supernatant was measured by BCA protein assay to determine insulin release, a blank and a positive control in which no TDM was added and the sample was not spun were also included. The data is shown in FIG. 2 and clearly shows that less than 5% insulin release by TDM over 0 TDM control (note y-axis break).

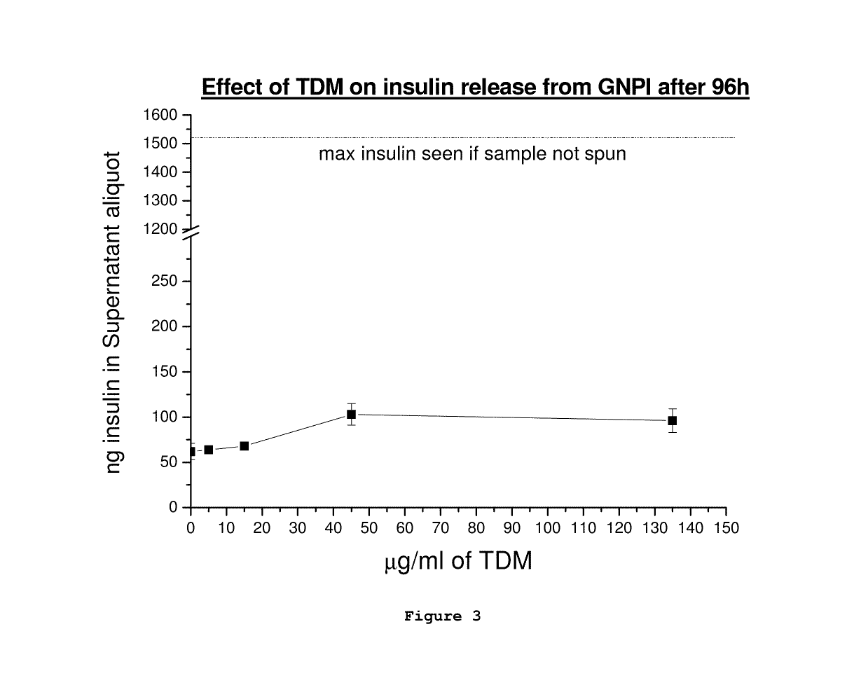

The sample tubes were vortexed and left at RT for a further 95 h (for a total of 96 hours) and re-assayed as above to see if there was any further indication of insulin release. The data are shown in FIG. 3, which suggests that there is no further release and that the low variations in responses we are seeing are probably within assay noise.

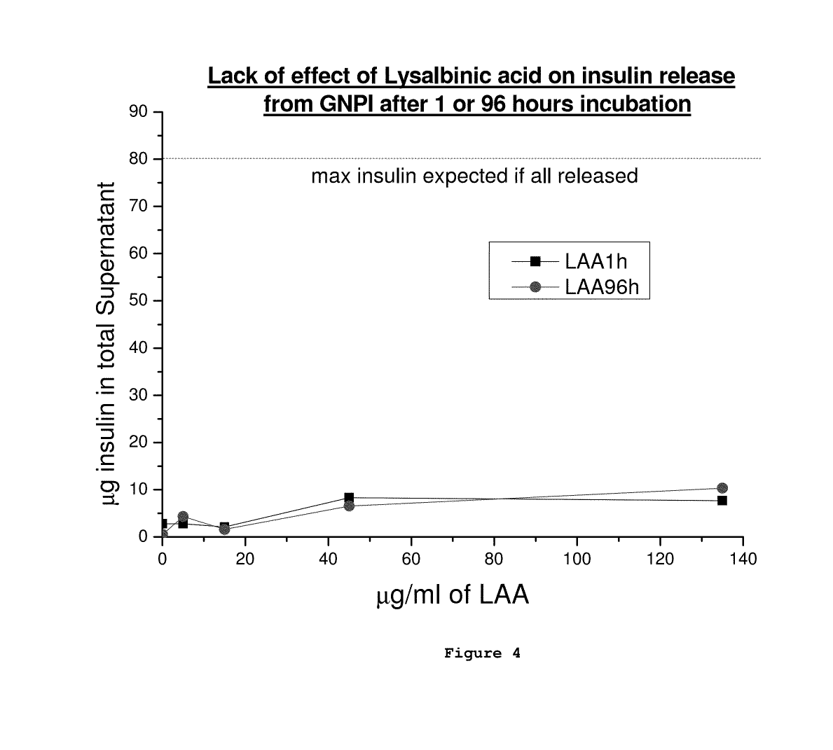

In a follow on experiment lysalbinic acid (LAA) was also tested as an alternative potential permeation enhancer, the same protocol was used as for TDM above. The data are shown in FIG. 4 and, although measurable insulin levels are slightly higher, the data clearly show that LAA has little if any effect on insulin release from GNPI.

### Conclusion

Overall the data suggest that TDM and LAA have no gross effect at a range of concentrations on insulin release from GNPI in water in the short or medium term.

### Example 2

### Permeation Enhanced Nanoparticle Delivery of Insulin In Vivo

Minipigs were infused with somatostatin in order to suppress endogenous insulin secretion. They were then given a bolus injection of 0.33 gm/kg glucose. After thirty minutes the test item was given and the first order rate constant for glucose clearance (k) was calculated for the period of 40 to 60 minutes.

Test items included:
1. Strips containing 4 U insulin on nanoparticle applied transbuccal;
2. Strips containing 4 U insulin on nanoparticle dodecyl-β-D-maltoside applied transbuccal;
3. Subcutaneous (s.c.) nanoparticle-insulin (NP-insulin);
4. S.C. commercial Humalog® insulin.

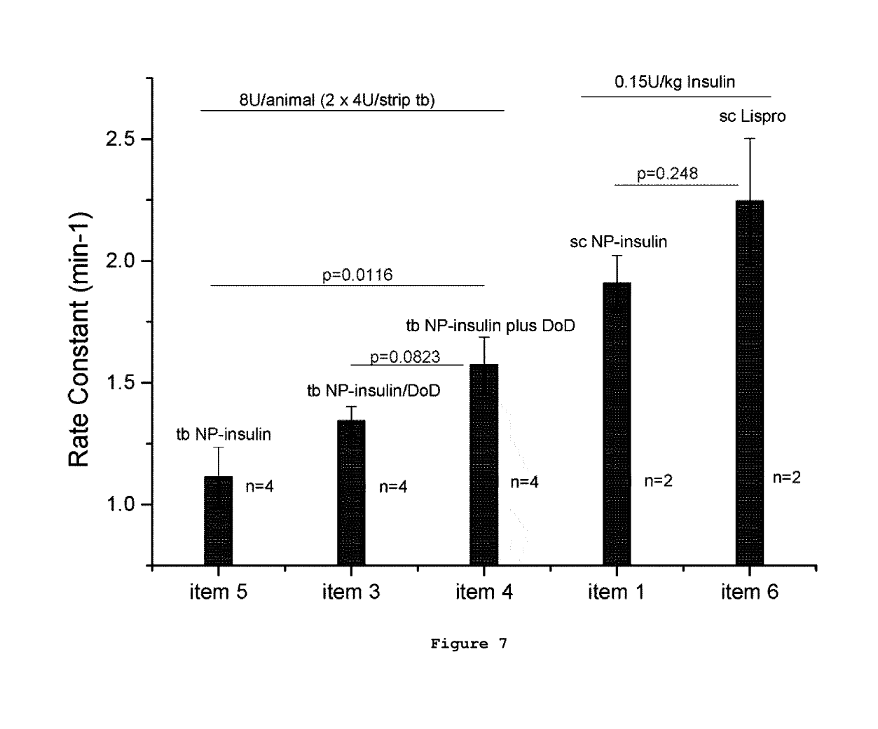

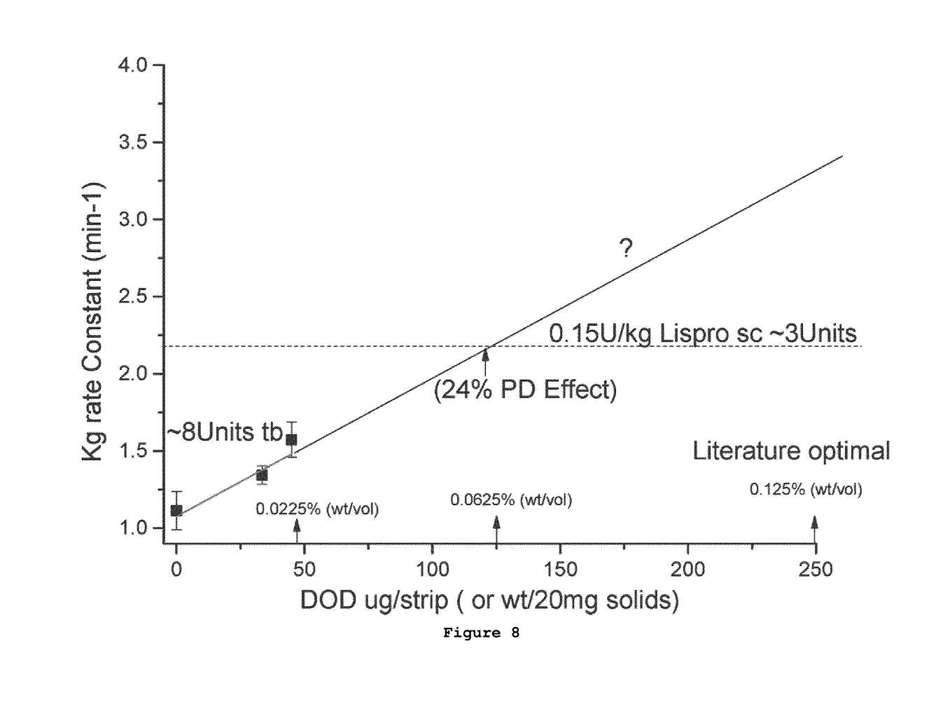

FIG. 7 shows the measured rate constants for the different test items. FIG. 8 shows an apparent dose-dependence for the rate constant for the inclusion of the dodecyl-β-D-maltoside (DoD).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1. A nanoparticle comprising:
(i) a core comprising a metal and/or a semiconductor;
(ii) a corona comprising a plurality of ligands covalently linked to the core, wherein at least one of said ligands comprises a carbohydrate moiety;
(iii) at least one peptide or other bioactive agent covalently or non-covalently bound to the corona; and
(iv) a permeation enhancer selected from: alkyl-D-maltoside and lysalbinic acid.

2. The nanoparticle according to claim 1, wherein the permeation enhancer is non-covalently bound to the corona.

3. The nanoparticle according to claim 1, wherein the nanoparticle comprises at least one peptide non-covalently bound to the corona.

4. The nanoparticle according to claim 1, wherein the peptide is selected from the group consisting of: insulin, GLP-1, IGF1, IGF2, relaxin, INSL5, INSL6, INSL7, pancreatic polypeptide (PP), peptide tyrosine tyrosine (PTT), neuropeptide Y, oxytocin, vasopressin, GnRH, TRH, CRH, GHRH/somatostatin, FSH, LH, TSH, CGA, prolactin, ClIP, ACTH, MSH, enorphins, lipotropin, GH, calcitonin, PTH, inhibin, relaxin, hCG, HPL, glucagons, somatostatin, melatonin, thymosin, thmulin, gastrin, ghrelin, thymopoietin, CCK, GIP secretin, motin VIP, enteroglucagon, IGF-1, IGF-2, leptin, adiponectin, resistin Osteocalcin, renin, EPO, calicitrol, ANP, BNP, chemokines, cytokines, adipokines and biologically active analogs thereof.

5. The nanoparticle according to claim 4, wherein the peptide is monomeric and/or dimeric human insulin.

6. The nanoparticle according to claim 1, wherein said at least one ligand comprising a carbohydrate moiety is selected from the group consisting of: 2′-thioethyl-α-D-galactopyranoside, 2′-thioethyl-β-D-glucopyranoside, 2′-thioethyl-2-acetamido-2-deoxy-β-D-glucopyranoside, 5′-thiopentanyl-2-deoxy-2-imidazolacetamido-α,β-D-glucopyranoside and 2′-thioethyl-α-D-glucopyranoside, and wherein said at least one ligand comprising a carbohydrate moiety is covalently linked to the core via its sulphur atom.

7. The nanoparticle according to claim 1, wherein said plurality of ligands covalently linked to the core comprises at least a first ligand and a second ligand, wherein the first and second ligands are different.

8. The nanoparticle according to claim 7, wherein:
(a) said first ligand comprises 2′-thioethyl-α-D-galactopyranoside and said second ligand comprises 1-amino-17-mercapto-3,6,9,12,15,-pentaoxa-heptadecanol;
(b) said first ligand comprises 2′-thioethyl-β-D-glucopyranoside or 2′-thioethyl-α-D-glucopyranoside and said second ligand comprises 5′-thiopentanyl-2-deoxy-2-imidazolacetamido-α,β-D-glucopyranoside;
(c) said first ligand comprises 2′-thioethyl-β-D-glucopyranoside or 2′-thioethyl-α-D-glucopyranoside and said second ligand comprises 1-amino-17-mercapto-3,6,9,12,15,-pentaoxa-heptadecanol; or
(d) said first ligand comprises 2′-thioethyl-2-acetamido-2-deoxy-β-D-glucopyranoside and said second ligand comprises 1-amino-17-mercapto-3,6,9,12,15,-pentaoxa-heptadecanol,
and wherein said first and second ligands are covalently linked to the core via their respective sulphur atoms.

9. The nanoparticle according to claim 1, wherein at least 5 or more peptide molecules are bound per core.

10. The nanoparticle according to claim 1, wherein the core comprises a metal selected from the group consisting of: Au, Ag, Cu, Pt, Pd, Fe, Co, Gd, Zn or any combination thereof.

11. The nanoparticle according to claim 1, wherein the nanoparticle core has a diameter in the range of about 0.5 nm to about 50 nm.

12. The nanoparticle according claim 1, wherein the nanoparticle comprises a divalent component.

13. The nanoparticles according to claim 12, wherein said divalent component is selected from the group consisting of zinc, magnesium, copper, nickel, cobalt, cadmium, or calcium, and oxides and salts thereof.

14. The nanoparticle according to claim 1, wherein the nanoparticle comprises at least two different species of peptide bound to the corona.

15. The nanoparticle according to claim 14, wherein said at least two different species of peptide comprise insulin and GLP-1.

16. The nanoparticle according claim 1, wherein said permeation enhancer comprises an alkyl maltoside selected from the group consisting of: dodecyl-β-D-maltoside, tetradecyl-β-D-maltoside, hexyl-β-D-maltoside, octyl-β-D-maltoside, nonyl-β-D-maltoside, decyl-β-D-maltoside, undecyl-β-D-maltoside, tridecyl-β-D-maltoside, and hexadecyl-β-D-maltoside.

17. The nanoparticle according to claim 1, wherein said permeation enhancer comprises lysalbinic acid sodium salt.

18. A pharmaceutical or cosmetic composition comprising a plurality of nanoparticles of claim 1 and one or more pharmaceutically or cosmetically acceptable carriers or excipients.

19. A method of enhancing the cellular permeability of an active-carrying nanoparticle, comprising:
(a) providing an active-carrying nanoparticle comprising:
(i) a core which includes a metal and/or a semiconductor;
(ii) a corona including a plurality of ligands covalently linked to the core, wherein at least one of said ligands includes a carbohydrate moiety; and
(iii) at least one peptide or other bioactive agent covalently or non-covalently bound to the corona; and
(b) contacting the at least one active-carrying nanoparticle with a permeation enhancer selected from: alkyl-maltoside and lysalbinic acid under conditions which allow the permeation enhancer to bind to the corona of the nanoparticle.

20. The method according to claim 19, wherein said active-carrying nanoparticle is a peptide-carrying nanoparticle comprising at least one peptide non-covalently bound to the corona.

21. The method according to claim 19, wherein the conditions which allow the permeation enhancer to bind to the corona of the nanoparticle comprise: incubating an aqueous solution containing both the permeation enhancer and the active-carrying nanoparticle for at least 5 minutes at a temperature between 4° C. and 70° C.

22. The method of claim 19, wherein the method further comprises separating the active-carrying nanoparticle having said permeation enhancer bound thereto from excess permeation enhancer.

23. The method of claim 19, wherein said permeation enhancer comprises an alkyl maltoside selected from the group consisting of: dodecyl-β-D-maltoside, tetradecyl-β-D-maltoside, hexyl-β-D-maltoside, octyl-β-D-maltoside, nonyl-β-D-maltoside, decyl-β-D-maltoside, undecyl-β-D-maltoside, tridecyl-β-D-maltoside, and hexadecyl-β-D-maltoside.

24. The method according to claim 19, wherein said permeation enhancer comprises lysalbinic acid sodium salt.

25. A method of lowering blood glucose or treating diabetes in a mammalian subject in need thereof, comprising administering a therapeutically effective amount of a nanoparticle of claim 1, wherein the peptide comprises insulin and/or GLP-1.

26. An article of manufacture comprising:
at least one nanoparticle of claim 1;
a container for housing the at least one nanoparticle; and
an insert and/or a label.
